(19) United States
(12) Patent Application Publication
(10) Pub. No.: US 2004/0171945 A1
(43) Pub. Date: Sep. 2, 2004

(54) Pulse wave velocity related information obtaining apparatus

(75) Inventor: Kiyoyuki Narimatsu, Komaki-shi (JP)

Correspondence Address: OLIFF & BERRIDGE, PLC, P.O. BOX 19928, ALEXANDRIA, VA, 22320, US

(73) Assignee: COLIN MEDICAL TECHNOLOGY CORPORATION, Komaki-shi (JP)

(21) Appl. No.: 10/780,770

(22) Filed: Feb. 19, 2004

(30) Foreign Application Priority Data
Feb. 28, 2003 (JP) 2003-053638

(51) Int. Cl. A61B 5/02

(52) U.S. Cl. 600/490; 600/493

(57) ABSTRACT

An apparatus including a brachium cuff 40 worn on a brachium of a patient, an ankle cuff 20 worn on an ankle of the patient, and a cuff pressure changing device 70 that slowly changes, in a blood pressure measuring operation, respective pressing pressures of the brachium and ankle cuffs 40, 20, while keeping the respective pressing pressures equal to each other. The apparatus additionally includes a pulse wave propagation velocity determining means 78 that determines a pulse wave propagation velocity PWV based on brachium and ankle pulse waves that are detected by the brachium and ankle cuffs 40, 20, respectively, when the respective pressing pressures of the brachium and ankle cuffs 40, 20 are equal to a systolic blood pressure of the brachium of the patient.

### BACKGROUND OF THE INVENTION

1. Field of the Invention

The present invention relates to a pulse wave velocity related information obtaining apparatus which obtains information related to a velocity at which a pulse wave propagates in a living subject.

2. Related Art Statement

Information related to a velocity at which a pulse wave propagates in a living subject (hereinafter, referred to as the “pulse wave velocity related information”) is a time difference between respective times of detection of respective periodic portions of two pulse waves, or two heartbeat synchronous waves, detected from two body portions of the subject, or information obtained based on the time difference. The pulse wave velocity related information is used in diagnosing various diseases. For example, Patent Document 1 (Japanese Patent Publication No. 9-140679) teaches that pulse wave velocity related information is used in diagnosing arteriosclerosis, because the information changes with hardening of artery; and Patent Document 2 (Japanese Patent Publication No. 7-31593) teaches that pulse wave velocity related information is used in estimating continuously blood pressure, because the information changes with the blood pressure.

Since the pulse wave velocity related information obtained from a living subject changes with the blood pressure of the subject, the information is used in diagnosing arteriosclerosis, while taking into account a blood pressure value of the subject at the time when the information is obtained from the subject. Meanwhile, in the case where the pulse wave velocity related information obtained from a living subject is used in estimating continuously the blood pressure of the subject, it is needed to determine a relationship between pulse wave velocity related information and blood pressure, and accordingly it is needed to obtain a blood pressure value of the subject at the time when the information is obtained from the subject.

Thus, there are many cases in which, when a diagnosis is made based on pulse wave velocity related information obtained from a living subject, a blood pressure value of the subject is also needed. However, the blood pressure of the subject always changes. Therefore, in the case where the pulse wave velocity related information and the blood pressure are obtained from the subject, it is preferred to obtain simultaneously the information and the blood pressure from the subject.

However, the accuracy of pulse wave velocity related information obtained based on the pulse waves detected during the blood pressure measuring operation is low because of the following reasons: In a state in which an artery is pressed by a cuff, the artery does not pulsate unless the inner pressure (i.e., the blood pressure) of the artery is higher than the pressing pressure of the cuff. Thus, the pulse wave detected when the blood pressure is measured using the cuff does not contain a low-pressure component because of the pressing of the cuff. A pulse wave is a composite wave consisting of an incident wave that is produced when blood is ejected from the left ventricle of the heart of a living person and advances toward a peripheral body portion of the person; a primary reflected wave produced when the incident wave is reflected by a peripheral, bifurcated portion of the artery and advances toward the heart; and a secondary reflected wave produced when the primary reflected wave is reflected by the aortic valve of the heart. As the times of reflection of the pulse wave increase, the pressure of the reflected wave decreases. Therefore, when the low-pressure component is lost from the pulse wave by the pressing of the cuff, the low-pressure components corresponding to the higher-order reflected waves are lost earlier. Thus, if the low-pressure component is lost from the pulse wave because of the pressing of the cuff, the shape or waveform of the pulse wave changes and accordingly the maximum point (i.e., peak point) of the pulse wave changes. Meanwhile, an amount of lost of low-pressure component from a pulse wave depends on a relationship between pressing pressure of cuff and inner pressure of artery pressed by the cuff. Since, however, the pressing pressure of the cuff slowly changes during the blood pressure measuring operation and the blood pressure of the artery always changes, the amount of lost of low-pressure component also changes. Thus, during the blood pressure measuring operation, the reference point, such as the maximum point, on the pulse wave that is used to obtain the pulse wave velocity related information changes because of the change of pressing pressure of the cuff and the change of blood pressure of the artery. Therefore, there has been the problem that the accuracy of pulse wave velocity related information obtained based on the pulse waves detected during the blood pressure measuring operation is insufficiently low.

To solve this problem, Patent Document 2 proposes to detect pulse waves to be used to obtain pulse wave velocity related information, at times as near as possible to a time when blood pressure is measured using a cuff, within a range in which the pulse waves detected are not influenced by the pressing of the cuff, more specifically described, proposes to detect the pulse waves immediately before or after the commencement of pressing of the cuff in the blood pressure measuring operation, and calculate a pulse wave propagation time based on the thus detected pulse waves.

However, blood pressure of a living person may largely change in a very short time. Therefore, to make a more accurate diagnosis, it is desirable to obtain more accurate pulse wave velocity related information based on pulse waves detected at times as near as possible to the time of measurement of blood pressure.

### SUMMARY OF THE INVENTION

It is therefore an object of the present invention to provide a pulse wave velocity related information obtaining apparatus which can obtain accurate pulse wave velocity related information based on pulses detected during a blood pressure measuring operation.

The above object has been achieved according to the present invention. According to the present invention, there is provided an apparatus for obtaining pulse wave velocity related information that is related to a velocity at which a pulse wave propagates in a living subject, the apparatus comprising a first cuff which is adapted to be worn on a first portion of the subject, and detects a first pulse wave from the first portion; a second cuff which is adapted to be worn on a second portion of the subject that is distant from the first portion, and detects a second pulse wave from the second portion; a cuff pressure changing means for changing respective pressing pressures of the first and second cuffs, while keeping the respective pressing pressures of the first and second cuffs equal to each other; and a pulse wave velocity related information obtaining means for obtaining the pulse wave velocity related information based on a time difference between respective prescribed points on the first and second pulse waves respectively detected by the first and second cuffs when the respective pressing pressures of the first and second cuffs are changed by the cuff pressure changing means such that the respective pressing pressures of the first and second cuffs are kept equal to each other.

According to the present invention, the pulse wave velocity related information obtaining means obtains the pulse wave velocity related information, by using the first pulse wave that is detected during the changing of pressing pressure of the first cuff and accordingly does not contain the low-pressure component because of the pressing pressure. However, the cuff pressure changing means keeps, during the changing of the pressing pressure of the first cuff, the respective pressing pressures of the first and second cuffs equal to each other. Thus, the second pulse wave used to obtain the pulse wave velocity related information does not contain the low-pressure component, either. Thus, the accuracy of pulse wave velocity related information is less influenced by the loss of respective low-pressure components of the first and second pulse waves. Therefore, accurate pulse wave velocity related information can be obtained based on pulse waves detected in a blood pressure measuring operation.

Here, preferably, the pulse wave velocity related information obtaining means obtains the pulse wave velocity related information based on the first and second pulse waves respectively detected by the first and second cuffs when the respective pressing pressures of the first and second cuffs are equal to a blood pressure of the first portion of the subject. According to this feature, the pulse wave velocity related information is obtained based on the first and second pulse waves detected when the pressing pressure of the first cuff is equal to the blood pressure of the first portion of the subject. Thus, accurate pulse wave velocity related information when the pressing pressure of the first cuff equal to the blood pressure of the first portion is detected, can be obtained.

Also, preferably, the pulse wave velocity related information obtaining means obtains the pulse wave velocity related information based on the first and second pulse waves respectively detected by the first and second cuffs when the respective pressing pressures of the first and second cuffs are equal to a systolic, a mean, or diastolic blood pressure of the first portion of the subject. Since, however, the systolic or diastolic blood pressure is important, it is preferred to obtain the pulse wave velocity related information based on the first and second pulse waves respectively detected by the first and second cuffs when the respective pressing pressures of the first and second cuffs are equal to the systolic or diastolic blood pressure of the first portion of the subject.

### BRIEF DESCRIPTION OF THE DRAWINGS

The above and optional objects, features, and advantages of the present invention will be better understood by reading the following detailed description of the preferred embodiments of the invention when considered in conjunction with the accompanying drawings, in which:

FIG. 1 is a diagrammatic view for explaining a construction of an arteriosclerosis diagnosing apparatus functioning as a pulse wave propagation velocity related information obtaining apparatus to which the present invention is applied;

FIG. 2 is a diagrammatic view for explaining essential control functions of an electronic control device of the apparatus of FIG. 1;

FIG. 3 is a two dimensional graph that is defined by a first axis indicative of brachium cuff pressure and a second axis indicative of amplitude of brachium pulse wave, and shows an example of an array of respective amplitudes of successive heartbeat synchronous pulses of a brachium pulse wave detected by a brachium cuff of the apparatus of FIG. 1;

FIG. 4 is a flow chart representing a cuff pressure changing function and a blood pressure measuring function as a part of the essential control functions of the electronic control device, shown in FIG. 2;

FIG. 5 is another flow chart representing the cuff pressure changing function and the blood pressure measuring function of the electronic control device;

FIG. 6 is a view for explaining respective changes of cuff pressures PC_{A}, PC_{B} that are changed according to the flow charts of FIGS. 4 and 5; and

FIG. 7 is another flow chart representing another part of the essential control functions of the electronic control device, shown in FIG. 2, that is carried out after the flow charts of FIGS. 4 and 5.

### DETAILED DESCRIPTION OF PREFERRED EMBODIMENTS

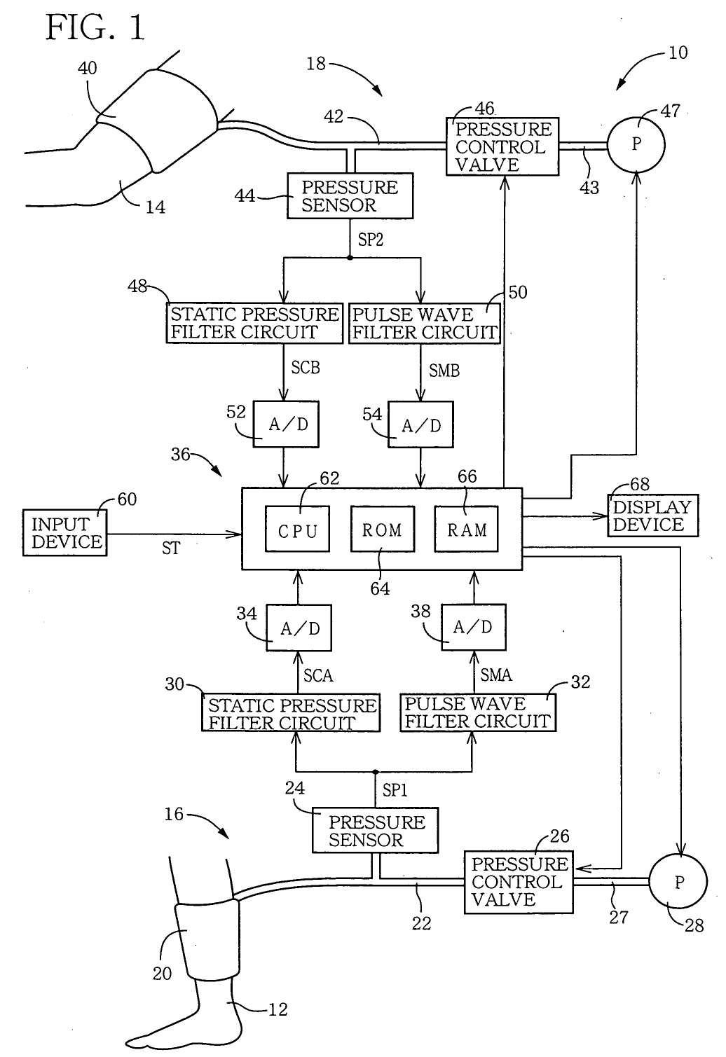

Hereinafter, there will be described a preferred embodiment of the present invention in detail by reference to the drawings. FIG. 1 is a view for explaining a construction of an arteriosclerosis diagnosing apparatus 10 functioning as a pulse wave propagation velocity related information obtaining apparatus to which the present invention is applied. The arteriosclerosis diagnosing apparatus 10 measures or calculates, as physical information for use in diagnosing arteriosclerosis, a pulse wave propagation velocity PWV at which a pulse wave propagates between two body portions of a patient as a living subject; a brachium blood pressure BP(B) as a blood pressure BP of a brachium 14 of the patient; an ankle blood pressure BP(A) as a blood pressure BP of an ankle 12 of the patient; and an ankle and brachium blood pressure index ABI of the patient. The diagnosing apparatus 10 displays the measured values and the calculated index. The diagnosing apparatus 10 performs those measurements in a state in which the patient takes a face-up position or a lateral position, so that the brachium 14 and the ankle 12 of the patient are substantially level with each other.

In FIG. 1, the arteriosclerosis diagnosing apparatus 10 includes an ankle blood pressure measuring device 16 which measures a blood pressure of the ankle 12 and functions as an inferior limb blood pressure measuring device; and a brachium blood pressure measuring device 18 which measures a blood pressure of the brachium 14 and functions as a superior limb blood pressure measuring device.

The ankle blood pressure measuring device 16 includes an ankle cuff 20 which is adapted to be wound around the ankle 12 of the patient and functions as a second cuff (a first cuff will be described below); a pressure sensor 24 and a pressure control valve 26 which are connected to the ankle cuff 20 via a piping 22; and an air pump 28 which is connected to the pressure control valve 26 via a piping 27. The ankle cuff 20 includes a belt-like cloth bag and a rubber bag accommodated in the cloth bag. The pressure control valve 26 adjusts a pressure of a pressurized air supplied from the air pump 28, and supplies the pressure adjusted air to the ankle cuff 20, or discharges the pressurized air from the ankle cuff 22, so as to control an air pressure in the ankle cuff 20.

The pressure sensor 24 detects the air pressure in the ankle cuff 20, and supplies a pressure signal, SP1, representing the detected air pressure, to a static pressure filter circuit 30 and a pulse wave filter circuit 32. The static pressure filter circuit 30 includes a low pass filter which extracts, from the pressure signal SP1, an ankle cuff pressure signal, SC_{A}, representing a static component of the detected air pressure, i.e., a pressing pressure of the ankle cuff 20 (hereinafter, referred to as the ankle cuff pressure, PC_{A}). The filter circuit 30 supplies the ankle cuff pressure signal SC_{A} to an electronic control device 36 via an A/D (analog to digital) converter 34.

The pulse wave filter circuit 32 includes a band pass filter which extracts, from the pressure signal SP1, an ankle pulse wave signal, SM_{A}, representing an ankle pulse wave as an oscillatory component of the detected air pressure that has prescribed frequencies. The filter circuit 32 supplies the ankle pulse wave signal SM_{A} to the control device 36 via an A/D converter 38. Since, in the present embodiment, the ankle pulse wave is detected by the ankle cuff 20 as the second cuff, the ankle pulse wave is a second pulse wave (a first pulse wave will be described below).

The brachium blood pressure measuring device 18 includes a brachium cuff 40 which is adapted to be wound around the brachium 14 and functions as the first cuff, and additionally includes a pressure sensor 44, a pressure control valve 46, an air pump 47, a static pressure filter circuit 48, and a pulse wave filter circuit 50 which have respective constructions identical with those of the counterparts of the ankle blood pressure measuring device 16. The brachium cuff 40 is connected to the pressure sensor 44 and the pressure control valve 46 via a piping 42; and the pressure control valve 46 is connected to the air pump 47 via a piping 43.

The pressure sensor 44 detects an air pressure in the brachium cuff 40, and supplies a pressure signal, SP2, representing the detected air pressure, to the static pressure filter circuit 48 and the pulse wave filter circuit 50. The static pressure filter circuit 48 extracts, from the pressure signal SP2, a brachium cuff pressure signal, SC_{B}, representing a static component of the detected air pressure, i.e., a pressing pressure of the brachium cuff 40 (hereinafter, referred to as the brachium cuff pressure, PC_{B}). The filter circuit 48 supplies the brachium cuff pressure signal SC_{B} to the control device 36 via an A/D converter 52. The pulse wave filter circuit 50 extracts, from the pressure signal SP2, a brachium pulse wave signal, SM_{B}, representing a brachium pulse wave as an oscillatory component of the detected air pressure that has prescribed frequencies. The filter circuit 50 supplies the brachium pulse wave signal SM_{B} to the control device 36 via an A/D converter 54. Since, in the present embodiment, the brachium pulse wave is detected by the brachium cuff 20 as the first cuff, the brachium pulse wave is the first pulse wave.

An input device 60 includes a plurality of numeral keys, not shown, which are manually operable for inputting numerals representing a stature T of the patient, and supplies a stature signal ST representing the patient's stature T inputted through the keys, to the electronic control device 36.

The electronic control device 36 is essentially provided by a microcomputer including a CPU (central processing unit) 62, a ROM (read only memory) 64, a RAM (random access memory) 66, and an I/O (input and output) port, not shown, and the CPU 62 processes signals according to control programs pre-stored in the ROM 64, while utilizing a temporary storage function of the RAM 66. The CPU 62 outputs, from the I/O port, drive signals to the two air pumps 28, 47 and the pressure control valves 26, 46 so as to control the respective operations thereof and thereby control the respective air pressures of the ankle cuff 20 and the brachium cuff 40. In addition, the CPU 62 processes signals supplied to the control device 36, so as to determine a brachium blood pressure BP(B), an ankle blood pressure BP(A) of the patient, a pulse wave propagation velocity PWV, and an ankle and brachium blood pressure index ABI of the patient, and controls a display device 68 to display the thus determined values.

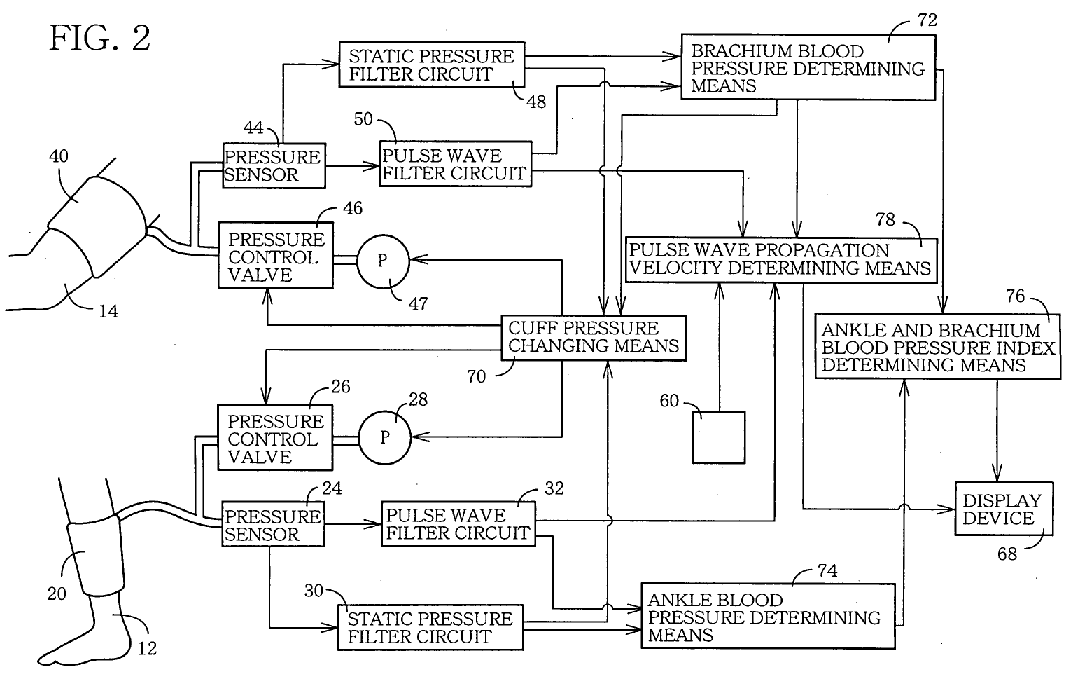

FIG. 2 is a diagrammatic view for explaining essential control functions of the electronic control device 36. A cuff pressure changing device or means 70 controls, according to a command signal supplied from a brachium blood pressure determining device or means 72, described later, and based on the ankle cuff pressure signal SC_{A} supplied from the static pressure filter circuit 30 and the brachium cuff pressure signal SC_{B} supplied from the static pressure filter circuit 48, the two air pumps 28, 47, and the two pressure control valves 26, 46 respectively connected to the two pumps 28, 47, so as to change the ankle cuff pressure PC_{A} and the brachium cuff pressure PC_{B}, as follows: First, the changing means 70 quickly increases the ankle cuff pressure PC_{A} UP to a prescribed first target pressure PC_{M1} (e.g., 240 mmHg) which would be higher than a systolic blood pressure BP(A)_{SYS} of the ankle 12, and then quickly increases the brachium cuff pressure PC_{B}up to a prescribed second target pressure PC_{M2} (e.g., 180 mmHg) which would be higher than a systolic blood pressure BP(B)_{SYS} of the brachium 14. Subsequently, the changing means 70 starts slowly decreasing the ankle cuff pressure PC_{A} and, when the ankle cuff pressure PC_{A} decreases down to the second target pressure PC_{M2}, the changing means 70 starts slowly decreasing the brachium cuff pressure PC_{B}. Thus, the changing means 70 slowly decreases the two cuff pressures PC_{A}, PC_{B}, while keeping the two cuff pressures PC_{A}, PC_{B} equal to each other. Finally, after determination of respective diastolic blood pressure values BP(A)_{DIA}, BP(B)_{DIA} of the ankle 12 and the brachium 14, the changing means 70 releases the ankle cuff pressure PC_{A} and the brachium cuff pressure PC_{B} to atmospheric pressure.

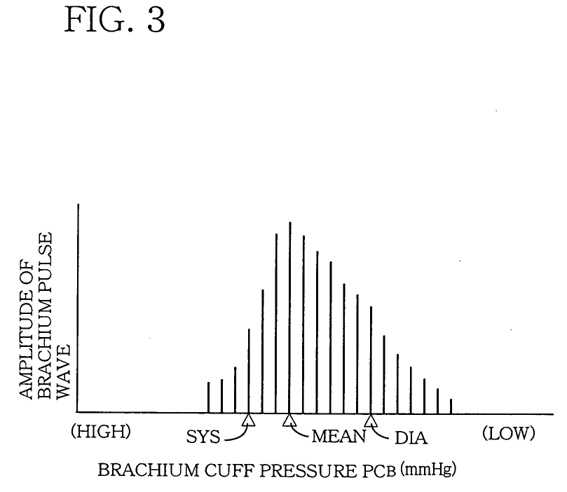

A brachium blood pressure determining device or means 72 determines, based on change of respective amplitudes of successive heartbeat-synchronous pulses of the brachium pulse wave that is continuously detected when the brachium cuff pressure PC_{B}is slowly decreased by the cuff pressure changing means 70, blood pressure values of the brachium 14, i.e., a brachium systolic blood pressure BP(B)_{SYS}, a brachium mean blood pressure BP(B)_{MEAN}, and a brachium diastolic blood pressure BP(B)_{DIA}, according to well known osciliometric algorithm. More specifically described, according to the oscillometric algorithm, first, an array of pulse amplitudes, as shown in FIG. 3, is obtained, then an envelope connecting respective upper ends of the pulse amplitudes is obtained, and finally a brachium cuff pressure PC_{B}corresponding to a rising point on the envelope is determined as a brachium systolic blood pressure BP(B)_{SYS}, a brachium cuff pressure PC_{B}corresponding to a maximum point on the envelope is determined as a brachium mean blood pressure BP(B)_{MEAN}, and a brachium cuff pressure PC_{B}corresponding to an inflection point on a curve obtained by differentiating the envelope is determined as a brachium diastolic blood pressure BP(B)_{DIA}. The inflection point on the curve obtained by differentiating the envelope corresponds to a falling point on the envelope.

An ankle blood pressure determining device or means 74 determines, based on change of respective amplitudes of successive heartbeat-synchronous pulses of the ankle pulse wave that is continuously detected when the ankle cuff pressure PC_{A}is slowly decreased by the cuff pressure changing means 70, blood pressure values of the ankle 12, i.e., an ankle systolic blood pressure BP(A)_{SYS}, an ankle mean blood pressure BP(A)_{MEAN}, and an ankle diastolic blood pressure BP(A)_{DIA}, according to the same oscillometric algorithm as used by the brachium blood pressure determining means 72.

An ankle and brachium blood pressure index determining device or means 76, functioning as an inferior and superior limb blood pressure index determining device or means, determines an ankle and brachium blood pressure index ABI of the patient, based on the ankle blood pressure BP(A) determined by the ankle blood pressure determining means 74, and the brachium blood pressure BP(B) that is determined by the brachium blood pressure determining means 72 and corresponds to the sort of ankle blood pressure BP(A) determined. For example, if the systolic ankle blood pressure BP(A)_{SYS} determined by the ankle blood pressure determining means 74 is used, the systolic brachium blood pressure BP(B)_{SYS} determined by the brachium blood pressure determining means 72 is used with the systolic ankle blood pressure BP(A)_{SYS} to determine the ankle and brachium blood pressure index ABI of the patient. The ankle and brachium blood pressure index ABI may be determined by dividing the ankle blood pressure BP(A) by the brachium blood pressure BP(B), or dividing the brachium blood pressure BP(B) by the ankle blood pressure BP(A). The determining means 76 operates the display device 68 to display the thus determined ankle and brachium blood pressure index ABI.

A pulse wave propagation velocity determining device or means 78, functioning as a pulse wave propagation velocity related information obtaining device or means, determines respective prescribed characteristic points (e.g., respective maximum points) of respective heartbeat synchronous pulses of the ankle pulse wave and the brachium pulse wave that are respectively detected by the ankle cuff 20 and the brachium cuff 40 (i.e., the ankle pulse wave signal SM_{A}and the brachium pulse wave signal SM_{B}that are respectively supplied from the pulse wave filter circuit 32 and the pulse wave filter circuit 50) while the ankle cuff pressure PC_{A}and the brachium cuff pressure PC_{B}are concurrently and slowly decreased, and additionally determines a time difference between the thus determined respective prescribed characteristic points of the respective pulses. The respective prescribed characteristic points of the respective pulses correspond to each other. For example, if the maximum point of pulse of one of the ankle and brachium pulse waves is determined, then the maximum point of pulse of the other pulse wave is determined. The thus determined time difference is a difference between a time needed for the ankle pulse wave to propagate from the patient's heart to the ankle 12 and a time needed for the brachium pulse wave to propagate from the patient's heart to the brachium 14, and means a pulse wave propagation time DT with respect to the ankle 12 and the brachium 14.

The respective heartbeat synchronous pulses of the ankle and brachium pulse waves that are used to determine the pulse wave propagation velocity PWV may be any pulses that are obtained at any time while the ankle cuff pressure PC_{A}and the brachium cuff pressure PC_{B}are concurrently and slowly decreased. However, in the case where a diagnosis is made based on the pulse wave propagation velocity PWV, it is desirable to obtain the blood pressure of the patient at the time when the propagation velocity PWV is determined. Hence, it is preferred to use the pulse wave propagation velocity PWV that is determined when one of the brachium systolic, mean, and diastolic blood pressure values BP(B)_{SYS}, BP(B)_{MEAN}, BP(B)_{DIA} and the ankle systolic, mean, and diastolic blood pressure values BP(A)_{SYS}, BP(A)_{MEAN}, BP(A)_{DIA} is determined. In particular, the brachium systolic blood pressure BP(B)_{SYS} is used in making a diagnosis in many cases. Thus, in the present embodiment, the pulse wave propagation velocity PWV is determined based on the respective heartbeat synchronous pulses of the ankle and brachium pulse waves that are obtained at the time when the brachium systolic blood pressure BP(B)_{SYS} is determined. Since, in the present embodiment, the blood pressure values are determined according to the oscillometric algorithm, the time when the brachium systolic blood pressure BP(B)_{SYS} is determined, means the rising point on the envelope shown in FIG. 3.

In addition, the pulse wave propagation velocity determining means 78 replaces the following expression (1) defining a relationship between stature T and distance difference L, with the patient's stature T supplied from the input device 60, and thereby determines a distance difference L between a first propagation distance from the patient's heart to the ankle 12 and a second propagation distance from the patient's heart to the brachium 14, and subsequently replaces the following expression (2) with the thus determined distance difference L and the above described pulse wave propagation time DT, and thereby determines a pulse wave propagation velocity PWV (cm/sec). Finally, the determining means 78 operates the display device 68 to display the thus determined pulse wave propagation velocity PWV:

L=aT+b Expression (1)

where a, b are experimentally obtained constants,

PWV=L/DT Expression (2)

Here, the accuracy of pulse wave propagation velocity PWV determined by the pulse wave propagation velocity determining means 78 is explained in detail. In the state in which the brachium cuff pressure PC_{B}is equal to the brachium systolic blood pressure BP(B)_{SYS}, the brachium pulse wave detected by the brachium cuff 40 does not contain a low-pressure component, because the brachium cuff pressure PC_{B}is high. Thus, this brachium pulse wave has a shape or waveform different from that of a brachium pulse wave detected in a state in which the brachium cuff pressure PC_{B}is lower than the brachium diastolic blood pressure BP(B)_{DIA}. It is noted that in the state in which the brachium cuff pressure PC_{B}is equal to the brachium systolic blood pressure BP(B)_{SYS}, a portion of the artery that is being pressed by the brachium cuff 40 does not pulsate, but pulsation of an upstream portion of the artery that is located upstream of the cuff 40 is transmitted to the cuff 40, so that a brachium pulse wave is detected by the cuff 40. Although the brachium pulse wave does not contain the low-pressure component, the ankle pulse wave does not contain a low-pressure component, either, because the ankle pulse wave is also detected in the state in which the ankle cuff pressure PC_{A}is equal to the brachium systolic blood pressure BP(B)_{SYS}. It is speculated that an amount of low-pressure component lost from a pulse wave depends on a relationship between cuff pressure and arterial pressure, and, usually, a brachium blood pressure BP(B) and an ankle blood pressure BP(A) of each individual patient differ from each other. Thus, usually, the amount of low-pressure component lost from the brachium pulse wave and the amount of low-pressure component lost from the ankle pulse wave are not equal to each other. However, since both the brachium and ankle pulse wave do not contain their low-pressure components, the pulse wave propagation velocity PWV is less influenced by the lost of low-pressure components. Therefore, although the two pulse waves detected by the two cuffs 20, 40 when the cuff pressures PC_{A}, PC_{B}are slowly decreased in the blood pressure measurement are used, the pulse wave propagation velocity PWV can be determined with accuracy based on those pulse waves.

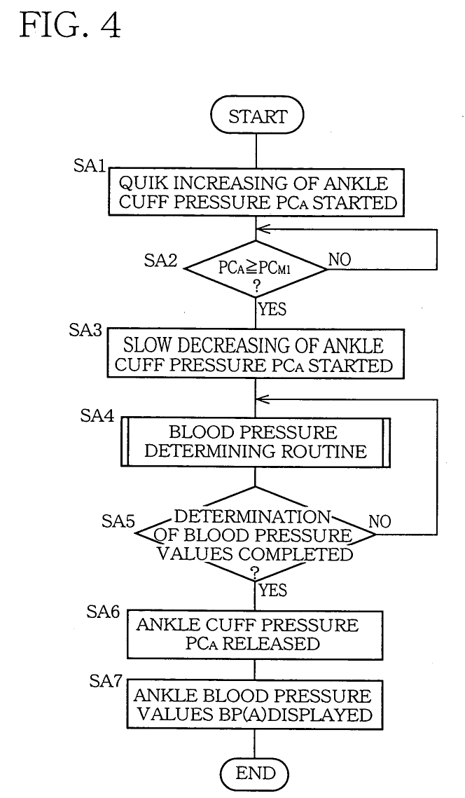

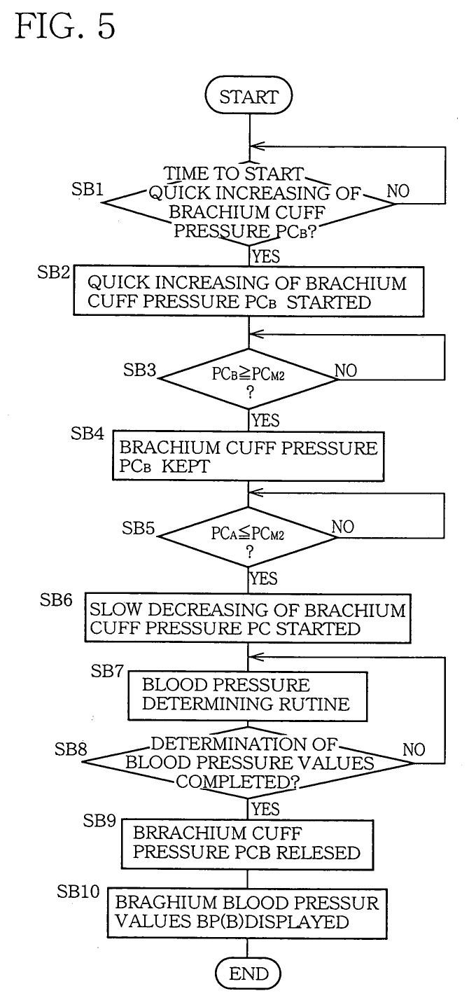

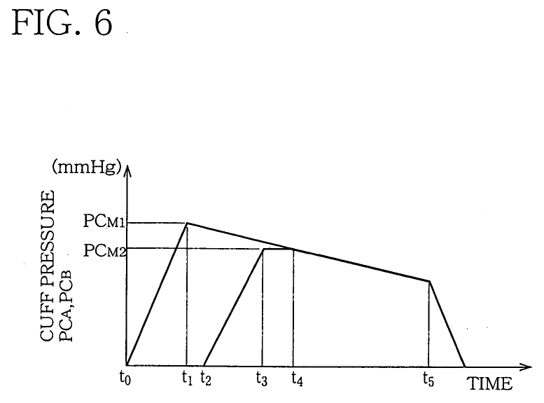

FIGS. 4 and 5 are respective flow charts representing the cuff pressure changing function, and the blood pressure measuring function, of the electronic control device 36, shown in FIG. 2; and FIG. 6 is a graph showing respective time-wise changes of cuff pressures PC_{A}, PC_{B}changed according to the flow charts of FIGS. 4 and 5. The routines shown in FIGS. 4 and 5 are started upon operation of a start button, not shown, under the condition that the stature signal ST representing the patient's stature T has already been supplied from the input device 60 to the control device 36. Those two routines are carried out concurrently by using well-known interruption or time division technique.

First, the routine of FIG. 4 is explained. At Step SA1, the control device 36 controls the air pump 28 and the pressure control valve 26 so as to start quick increasing of the ankle cuff pressure PC_{A}. This is a time, t0, shown in FIG. 6.

Subsequently, at Step SA2, the control device 36 judges whether the ankle cuff pressure PC_{A}has been increased up to the first target pressure PC_{M1}, e.g., 240 mmHg. Step SA2 is repeated till a positive judgment is made, while the quick increasing of the ankle cuff pressure PC_{A}is continued. Meanwhile, if a positive judgment is made at Step SA2, the control goes to Step SA3 to stop the air pump 28 and controls the pressure control valve 26 so as to start slow decreasing of the ankle cuff pressure PC_{A}, e.g., at a prescribed rate of 5 mmHg/sec. This is a time, t1, shown in FIG. 6.

Subsequently, at Step SA4, the control device 36 carries out a blood pressure determining routine. More specifically described, the control device 36 stores the ankle cuff pressure signal SC_{A} and the ankle pulse wave signal SM_{A}respectively supplied from the static pressure filter circuit 30 and the pulse wave filter circuit 32, determines respective values of the ankle cuff pressure PC_{A}represented by the ankle cuff pressure signal SC_{A}and respective amplitudes of successive heartbeat synchronous pulses of the ankle pulse wave represented by the ankle pulse wave signal SM_{A}, and determines, based on the thus determined respective values of the ankle cuff pressure PC_{A}and the thus determined respective amplitudes of successive heartbeat synchronous pulses of the ankle pulse wave, an ankle systolic blood pressure BP(A)_{SYS}, an ankle mean blood pressure BP(A)_{MEAN}, and an ankle diastolic blood pressure BP(A)_{DIA} of the patient, according to a well-known oscillometric blood pressure determining algorithm. Then, at Step SA5, the control device 36 judges whether the determination of ankle blood-pressure values BP(A) at Step SA4 has been completed, i.e., whether all the ankle systolic blood pressure BP(A)_{SYS}, ankle mean blood pressure BP(A)_{MEAN}, and ankle diastolic blood pressure BP(A)_{DIA} have been determined. If a negative judgment is made at Step SA5, the control device 36 repeats Step SA4 and the following steps.

Meanwhile, if a positive judgment is made at Step SA5, the control goes to Step SA6 so as to control the pressure control valve 26 to release the ankle cuff pressure PC_{A}to atmospheric pressure, thereby finishing the pressing of the ankle 12 with the ankle cuff 20. This is a time, t5, shown in FIG. 6. Subsequently, at Step SA7, the control device 36 operates the display device 68 to display the ankle systolic blood pressure BP(A)_{SYS}, the ankle mean blood pressure BP(A)_{MEAN}, and the ankle diastolic blood pressure BP(A)_{DIA}, all determined at Step SA4. In the embodiment shown in FIG. 4, Steps SA4, SA5, and SA7 correspond to the ankle blood pressure determining means 74.

Next, the routine of FIG. 5 is explained. First, at Step SB1, the control device 36 judges whether it is a time to start increasing of the brachium cuff pressure PC_{B}. More specifically described, the control device 36 judges whether a prescribed delay time has elapsed since the time t0 when the quick increasing of the ankle cuff pressure PC_{A}was started. This delay time is so prescribed as to assure that before the ankle cuff pressure PC_{A}is decreased down to the second target pressure PC_{M2}, e.g., 180 mmHg, the brachium cuff pressure PC_{B}can be increased up to the same pressure PC_{M2}, and can be kept at the same pressure PC_{M2} for as short as possible a time. If a negative judgment is made at Step SB1, the control device 36 repeats this step.

Meanwhile, if a positive judgment is made at Step SB1, the control proceeds with Step SB2 to control the air pump 47 and the pressure control valve 46 so as to start quick increasing of the brachium cuff pressure PC_{B}. This is a time, t2, shown in FIG. 6.

Subsequently, at Step SB3, the control device 36 judges whether the brachium cuff pressure PC_{B}has been increased up to the second target pressure PC_{M2}. Step SB3 is repeated till a positive judgment is made, while the quick increasing of the brachium cuff pressure PC_{B}is continued. Meanwhile, if a positive judgment is made at Step SB3, the control goes to Step SB4 to stop the air pump 47 and control the pressure control valve 46 so as to keep the brachium cuff pressure PC_{B}at the second target pressure PC_{M2}. This is a time, t3, shown in FIG. 6.

Subsequently, at Step SB5, the control device 36 judges whether the ankle cuff pressure PC_{A}has been decreased down to the second target pressure PC_{M2}. Step SB5 is repeated till a positive judgment is made, while the slow decreasing of the ankle cuff pressure PC_{A}is continued and the brachium cuff pressure PC_{B}is kept at the second target pressure PC_{M2}. Meanwhile, if a positive judgment is made at Step SB5, the control goes to Step SB6 to control the pressure control valve 46 so as to start slow decreasing of the brachium cuff pressure PC_{B}at the same rate as the rate of decreasing of the ankle cuff pressure PC_{A}. This is a time, t4, shown in FIG. 6.

Subsequently, at Step SB7, the control device 36 carries out a blood pressure determining routine. More specifically described, the control device 36 stores the brachium cuff pressure signal SC_{B} supplied from the static pressure filter circuit 48, and the brachium pulse wave signal SM_{B}supplied from the pulse wave filter circuit 50, determines respective values of the brachium cuff pressure PC_{B}represented by the brachium cuff pressure signal SC_{B} and respective amplitudes of successive heartbeat synchronous pulses of the brachium pulse wave represented by the brachium pulse wave signal SM_{B}, and determines, based on the thus determined respective values of the brachium cuff pressure PC_{B}and the thus determined respective amplitudes of successive heartbeat synchronous pulses of the brachium pulse wave, an brachium systolic blood pressure BP(B)_{SYS}, an brachium mean blood pressure BP(B)_{MEAN}, and an brachium diastolic blood pressure BP(B)_{DIA} of the patient, according to a well-known oscillometric blood pressure determining algorithm. Then, at Step SB8, the control device 36 judges whether the determination of brachium blood-pressure values at SB7 has been completed, i.e., whether all the brachium systolic blood pressure BP(B)_{SYS}, brachium mean blood pressure BP(B)_{MEAN}, and brachium diastolic blood pressure BP(B)_{DIA} have been determined. If a negative judgment is made at Step SB8, the control device 36 repeats Step SB7 and the following steps.

Meanwhile, if a positive judgment is made at Step SB8, the control goes to SB9 so as to control the pressure control valve 46 to release the brachium cuff pressure PC_{B}to the atmospheric pressure, thereby finishing the pressing of the brachium 14 with the brachium cuff 40. This is a time, t6, shown in FIG. 6. Subsequently, at Step SB10, the control device 36 operates the display device 68 to display the brachium systolic blood pressure BP(B)_{SYS}, the brachium mean blood pressure BP(B)_{MEAN}, and the brachium diastolic blood pressure BP(B)_{DIA}, all determined at Step SB7. In the embodiment shown in FIG. 5, Steps SB7, SB8, and SB10 correspond to the brachium blood pressure determining means 72. In the embodiment shown in FIGS. 4 and 5, Steps SA1 through SA3, SA6, SB1 through SB6, and SB9 correspond to the cuff pressure changing means 70.

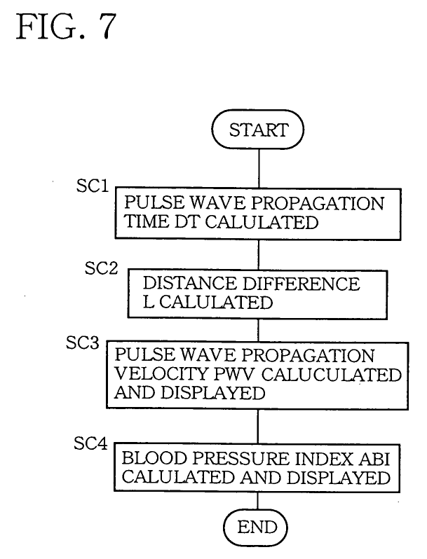

Following the routines shown in FIGS. 4 and 5, the control device 36 carries out the routine shown in FIG. 7. First, at Step SC1, the control device 36 determines respective maximum points (i.e., respective peak points) of respective heartbeat synchronous pulses of the ankle pulse wave and the brachium pulse wave that were detected when the ankle cuff pressure value PC_{A}equal to the ankle systolic blood pressure BP(A)_{SYS} determined at Step SA4, and the brachium cuff pressure value PC_{B}equal to the brachium systolic blood pressure BP(B)_{SYS} determined at Step SB7, were detected. In addition, the control device 36 determines, as a pulse wave propagation time DT, a time difference between respective times of detection of the thus determined maximum points.

Subsequently, at Step SC2, the control device 36 substitutes the above-indicated expression (1) with the patient's stature T represented by the stature signal ST supplied from the input device 40, so as to determine a distance difference L between a first distance between the patient's heart and the ankle 12 and a second distance between the patient's heart and the brachium 14. Then, at Step SC3, the control device 36 substitutes the above-indicated expression (2) with the pulse-wave propagation time DT determined at Step SC1 and the distance difference L determined at Step SC2, so as to calculate a pulse wave propagation velocity PWV (cm/sec). Finally, the control device 36 operates the display device 68 to display the thus calculated pulse wave propagation velocity PWV. In the flow chart shown in FIG. 7, Steps SC1 through SC3 correspond to the pulse wave propagation velocity determining means 78.

Subsequently, at Step SC4, the control device 36 calculates an ankle and brachium blood pressure index ABI of the patient, by dividing the ankle systolic blood pressure BP(A)_{SYS} determined at Step SA4 of FIG. 4, with the brachium systolic blood pressure BP(B)_{SYS} determined at Step SB7 of FIG. 5. In addition, the control device 36 operates the display device 68 to display the thus determined ankle and brachium blood pressure index ABI.

In the illustrated embodiment, the pulse wave propagation velocity determining means 78 (Steps SC1 through SC3) determines the pulse wave propagation velocity PWV by using the brachium pulse wave that is detected during the slow decreasing of pressing pressure of the brachium cuff 40 and accordingly does not contain the low-pressure component because of the pressing pressure. However, the cuff pressure changing means 70 (Steps SA1 through SA3, SA6, SB1 through SB6, and SB9) keeps, during the slow decreasing of pressing pressure of the brachium cuff 40, the respective pressing pressures of the brachium cuff 40 and the ankle cuff 20 equal to each other. Thus, the ankle pulse wave used to determine the pulse wave propagation velocity PWV does not contain the low-pressure component, either. Thus, the accuracy of pulse wave propagation velocity PWV is less influenced by the loss of respective low-pressure components of the brachium and ankle pulse waves. Therefore, an accurate pulse wave propagation velocity PWV can be determined based on pulse waves detected in a blood pressure measuring operation.

In the illustrated embodiment, in particular, the pulse wave propagation velocity PWV is determined based on the brachium and ankle pulse waves detected when the pressing pressure of the brachium cuff 40 is equal to the systolic blood pressure BP(B)_{SYS} of the brachium 14. Thus, an accurate pulse wave propagation velocity PWV when the brachium cuff pressure value PC_{B}equal to the systolic blood pressure BP(B)_{SYS} of the brachium 14 is detected, can be obtained.

While the present invention has been described in its embodiment by reference to the drawings, it is to be understood that the invention may otherwise be embodied.

For example, in the illustrated embodiment, the brachium cuff 40 provides the first cuff, and the ankle cuff 20 provides the second cuff. However, it may be vice versa, that is, the ankle cuff 20 may provide the first cuff, and the brachium cuff 40 may provide the second cuff. In addition, the body portions of the patient where the cuffs 20, 40 are worn are not limited to the ankle 12 and the brachium 14. One or both of the two cuffs may be worn on a femoral portion and/or a finger of the patient. Moreover, each of the cuffs worn on the femoral portion and the finger may provide either the first or second cuff.

In the illustrated embodiment, the respective reference points of the two pulse waves that are used to determine the pulse wave propagation velocity PWV (more specifically described, the pulse wave propagation time DT) are the respective maximum points (i.e., respective peak points) of respective heartbeat synchronous pulses of the two pulse waves. However, respective rising points of respective pulses of the two pulse waves may be used as the respective reference points of the two pulse waves.

It is to be understood that the present invention may be embodied with other changes, improvements and modifications that may occur to a person skilled in the art without departing from the spirit and scope of the invention defined in the appended claims.

What is claimed is:

1. An apparatus for obtaining pulse wave velocity related information that is related to a velocity at which a pulse wave propagates in a living subject, the apparatus comprising:
a first cuff which is adapted to be worn on a first portion of the subject, and detects a first pulse wave from the first portion;
a second cuff which is adapted to be worn on a second portion of the subject that is distant from the first portion, and detects a second pulse wave from the second portion;
a cuff pressure changing means for changing respective pressing pressures of the first and second cuffs, while keeping the respective pressing pressures of the first and second cuffs equal to each other; and
a pulse wave velocity related information obtaining means for obtaining the pulse wave velocity related information based on a time difference between respective prescribed points on the first and second pulse waves respectively detected by the first and second cuffs when the respective pressing pressures of the first and second cuffs are changed by the cuff pressure changing means such that the respective pressing pressures of the first and second cuffs are kept equal to each other.

2. The apparatus according to claim 1, wherein the pulse wave velocity related information obtaining means obtains the pulse wave velocity related information based on the first and second pulse waves respectively detected by the first and second cuffs when the respective pressing pressures of the first and second cuffs are equal to a blood pressure of the first portion of the subject.

3. The apparatus according to claim 2, wherein the pulse wave velocity related information obtaining means obtains the pulse wave velocity related information based on the first and second pulse waves respectively detected by the first and second cuffs when the respective pressing pressures of the first and second cuffs are equal to a systolic blood pressure of the first portion of the subject.

4. The apparatus according to claim 2, wherein the pulse wave velocity related information obtaining means obtains the pulse wave velocity related information based on the first and second pulse waves respectively detected by the first and second cuffs when the respective pressing pressures of the first and second cuffs are equal to a diastolic blood pressure of the first portion of the subject.

5. The apparatus according to claim 1, further comprising at least one blood pressure measuring device which measures a blood pressure of at least one of the first and second portions of the subject, and includes at least one of the first and second cuffs.

6. The apparatus according to claim 1, further comprising two blood pressure measuring devices which measure respective blood pressure of the first and second portions of the subject, and include the first and second cuffs, respectively.

7. The apparatus according to claim 1, wherein the pulse wave velocity related information obtaining means obtains, as the pulse wave velocity related information, at least one of said time difference and said velocity.

8. The apparatus according to claim 1, further comprising a display device which displays the pulse wave velocity related information obtained by the pulse wave velocity related information obtaining means.

9. The apparatus according to claim 1, further comprising:
an input device which is operable for inputting a stature of the subject; and
means for determining, based on the stature of the subject inputted through the input device, a distance between the first and second portions of the subject,
wherein the pulse wave velocity related information obtaining means obtains, as the pulse wave velocity related information, said velocity by dividing said distance by said time difference.

10. An apparatus for obtaining pulse wave velocity related information that is related to a velocity at which a pulse wave propagates in a living subject, the apparatus comprising:
a first cuff which is adapted to be worn on a first portion of the subject, and detects a first pulse wave from the first portion;
a second cuff which is adapted to be worn on a second portion of the subject that is distant from the first portion, and detects a second pulse wave from the second portion;
a cuff pressure changing device which changes respective pressing pressures of the first and second cuffs, while keeping the respective pressing pressures of the first and second cuffs equal to each other; and
a pulse wave velocity related information obtaining device which obtains the pulse wave velocity related information based on a time difference between respective prescribed points on the first and second pulse waves respectively detected by the first and second cuffs when the respective pressing pressures of the first and second cuffs are changed by the cuff pressure changing means such that the respective pressing pressures of the first and second cuffs are kept equal to each other.
